(19) United States
(12) Patent Application Publication
(10) Pub. No.: US 2011/0033509 A1
(43) Pub. Date: Feb. 10, 2011

(54) Cosmetic or dermatological article comprising a medium that is soluble in water

(75) Inventor: Pascal Simon, Thiais (FR)

Correspondence Address: OLIFF & BERRIDGE, PLC, P.O. BOX 320850, ALEXANDRIA, VA, 22320-4850, US

(73) Assignee: L'OREAL, Paris (FR)

(21) Appl. No.: 12/805,327

(22) Filed: Jul. 26, 2010

Related U.S. Application Data
(62) Division of application No. 11/303,961, filed on Dec. 19, 2005, now abandoned.
(60) Provisional application No. 60/658,165, filed on Mar. 4, 2005.

(30) Foreign Application Priority Data
Jan. 3, 2005 (FR) 05 50014

(51) Int. Cl. A61K 8/02; A61K 8/89; A61Q 19/00; A61Q 3/00; A61Q 5/00; A61Q 1/00

(52) U.S. Cl. 424/401; 424/70.1; 424/61; 424/78.02

(57) ABSTRACT

The present invention relates to a cosmetic or dermatological article comprising:
a medium in the form of at least one sheet of a material that is soluble in water at a temperature lower than 20° C.; and
at least one cosmetic or dermatological compound carried by the medium.

This is a Division of application Ser. No. 11/303,961 filed Dec. 19, 2005 which claims the benefit of French Application No. 05 50014 filed on Jan. 3, 2005 and U.S. Provisional Application No. 60/658,165 filed on Mar. 4, 2005. The entire disclosure of the prior applications are hereby incorporated by reference herein in their entirety.

The present invention relates to cosmetic or dermatological articles comprising at least one medium, and at least one cosmetic composition carried by the medium.

### BACKGROUND

Articles comprising a flexible medium that is insoluble in water, and a cosmetic composition that is carried by the medium, also known as “wipes”, are in widespread use, in particular for removing makeup.

Wet wipes that are impregnated with a latherable water-based composition are thus known from publications WO 02/092050, WO 02/092052, WO 02/092052, and U.S. Pat. No. 6,287,757, amongst others.

U.S. Pat. No. 4,303,543 discloses dry wipes that are impregnated with a latherable composition during manufacture, and then dried after impregnation.

Publication WO 00/07561 describes two-layer sponges containing a quantity of latherable powder.

Known wipes or sponges comprise a medium constituted by a substrate that is insoluble in water, and they are a source of solid waste.

U.S. Pat. No. 6,818,603 discloses a block of soap incorporating fibers which can be water-soluble.

U.S. Pat. No. 6,576,575 describes an adhesive dressing comprising fibers that are soluble in water.

### SUMMARY

In one of its aspects, the invention relates to a cosmetic or dermatological article comprising:
a medium in the form of at least one sheet of a material that is soluble in water at a temperature lower than 20° C.; and
at least one cosmetic or dermatological compound carried by the medium.

The terms “sheet” and “layer” should be understood as being synonymous.

In another of its aspects, the invention also provides a cosmetic or dermatological composition in fluid form that is obtained by dissolving, in water, a medium in the form of at least one sheet of a material that is soluble in water, and carrying at least one cosmetic or dermatological compound.

The term “fluid” should be understood as “able to flow under the effect of its own weight”.

In another of its aspects, the invention also provides a cosmetic or dermatological treatment comprising:
forming a cosmetic or dermatological composition by dissolving, in water, a medium comprising:
   a material that is soluble in water, in particular a sheet of such a material; and
   at least one cosmetic or dermatological compound carried by the medium; and
applying the composition formed in this way to the human body, e.g. to keratinous materials.

The cosmetic treatment may comprise applying makeup.

In another of its aspects, the invention also provides a cosmetic article comprising:
at least one cosmetic composition; and
at least one sheet of fibers that are soluble in water, preferably at a temperature lower than 20° C., the density of the sheet being not greater than 0.1 grams per cubic centimeter (g/cm³).

The invention also provides a cosmetic article comprising:
at least two layers defining a cavity between them, with at least one of the layers being soluble in water; and
at least one cosmetic composition inside the cavity.

In another of its aspects, the invention also provides a kit comprising:
packaging; and
at least one article as defined above. The packaging may include a colored indicator that is representative of the color of the composition.

In another of its aspects, the invention relates to a non-adhesive and preferably flexible cosmetic or dermatological article comprising:
at least one medium that is soluble in water, preferably completely soluble in water, and more preferably, soluble at a temperature lower than 20° C.; and
at least one cosmetic or dermatological compound carried by the medium.

The medium may carry a cosmetic or dermatological composition.

By way of example, the cosmetic composition is contained in the medium or secured thereon.

The term “soluble in water at a temperature lower than 20° C.” should be understood as dissolving in water at a temperature lower than 20° C. by manually agitating and/or rubbing the medium if necessary, over a period of time that is typically less than 5 minutes (min), preferably less than 1 min, and more preferably less than 30 seconds. The invention does not exclude water at a temperature that is higher than 20° C. from being used to dissolve the medium.

The term “flexible” should be understood as an article that is capable of being compressed or flexed without rupturing, and that is capable of being adapted to the contours of the human body. In some embodiments, a flexible article made in the form of a fibrous sheet may be folded in half at least once, without breaking into two pieces.

By way of example, the article is for single use only.

The invention may present the advantage of reducing the quantity of solid waste produced by the consumer.

An article made in accordance with the invention may also offer additional appeal associated with it changing its appearance in use as a result of the medium dissolving, and this may please young children in particular, and encourage them to wash their hands or clean their teeth more often or more thoroughly.

The invention may also offer novel possibilities for packaging and formulating numerous products, e.g. cosmetics, hygiene products, or care products for the skin, the lips, the mouth, or hair.

The invention may also make it possible to package cosmetic compositions in powder form that do not lend themselves well to conventional packaging methods, such as compacting them in a dish or incorporating them in a paste, for example.

The invention may also make it easier to package cosmetic compositions in paste or powder form that are difficult to take manually.

### BRIEF DESCRIPTION OF THE DRAWINGS

FIG. 1 is a plan view showing an article capable of retaining a cosmetic or dermatological composition in an internal cavity.

FIG. 2 is a cross section of the article of FIG. 1.

FIG. 3 is a diagrammatic and perspective view of a cosmetic article.

FIG. 4 is a fragmentary and diagrammatic cross-section of FIG. 3.

FIG. 5 is a plan view showing an article formed of a globular mass of compacted water-soluble fibers incorporating a cosmetic or dermatological composition.

FIG. 6 is a plan view showing an article having a medium that is of kidney-shaped outline.

FIG. 7 is a front view showing an article having a medium that forms a mask.

FIG. 8 is a diagram in perspective showing an article having a medium that forms a mobcap.

FIG. 9 is a diagram in section showing an article having a layer of a substrate that is insoluble in water, covered by a layer constituted by fibers that are soluble in water.

FIG. 10 is a diagram in perspective of an article for use in dental hygiene.

FIG. 11 shows a packaging for an article.

### EMBODIMENTS

### Medium

The medium may be in the form of one or more non-fibrous films that are obtained by evaporating a solvent, for example, but the medium preferably includes interlocking fibers that are soluble in water, preferably at a temperature lower than 20° C.

In an embodiment of the invention, the medium comprises at least one layer of a non-woven fabric that is constituted essentially by fibers that are soluble in water, preferably at a temperature lower than 20° C.

Once wet, the medium may be substantially non-shrink.

When the medium comprises a non-woven fabric, any technique that is suitable for making a non-woven fabric from fibers may be used in order to manufacture the medium.

For example, the fibers may be formed by extrusion and disposed on a conveyor so as to form a sheet of fibers that is then consolidated by a conventional fiber-bonding technique, e.g. needling, heat-bonding, calendering, or air through bonding, a technique in which the sheet passes into a tunnel into which hot air is injected. The air through bonding technique is advantageously used when the sheet is constituted by dual-component fibers, e.g. fibers comprising at least two grades of polyvinyl alcohol (PVA) having melting or softening points that are different, the fibers being co-extruded, for example, so that the fiber is constituted by at least one first grade located at the core of the fiber, and by at least one second grade located at the periphery of the fiber, in the form of a sheath. The fibers can thus be bonded more easily when the sheath presents a melting point that is lower than the core.

The sheet of fibers used for manufacturing the non-woven fabric can also be formed of carding fibers that are cut to a length lying in the range 10 millimeters (mm) to 50 mm, and then by depositing the fibers on a conveyor where the sheet can then be consolidated by a bonding technique as described above.

The density of the medium depends on its applications. The medium may present a density that is optionally not greater than 0.1 g/cm³, for example. A density that is not greater than 0.1 g/cm³, better in the range 0.01 g/cm³ to 0.1 g/cm³, can make it very loose, and make it easier to dissolve in water.

### DETAILED DESCRIPTION OF EMBODIMENTS

In an embodiment, the medium comprises at least two layers of non-woven fabric that are each constituted essentially by fibers that are soluble in water, preferably at a temperature lower than 20° C.

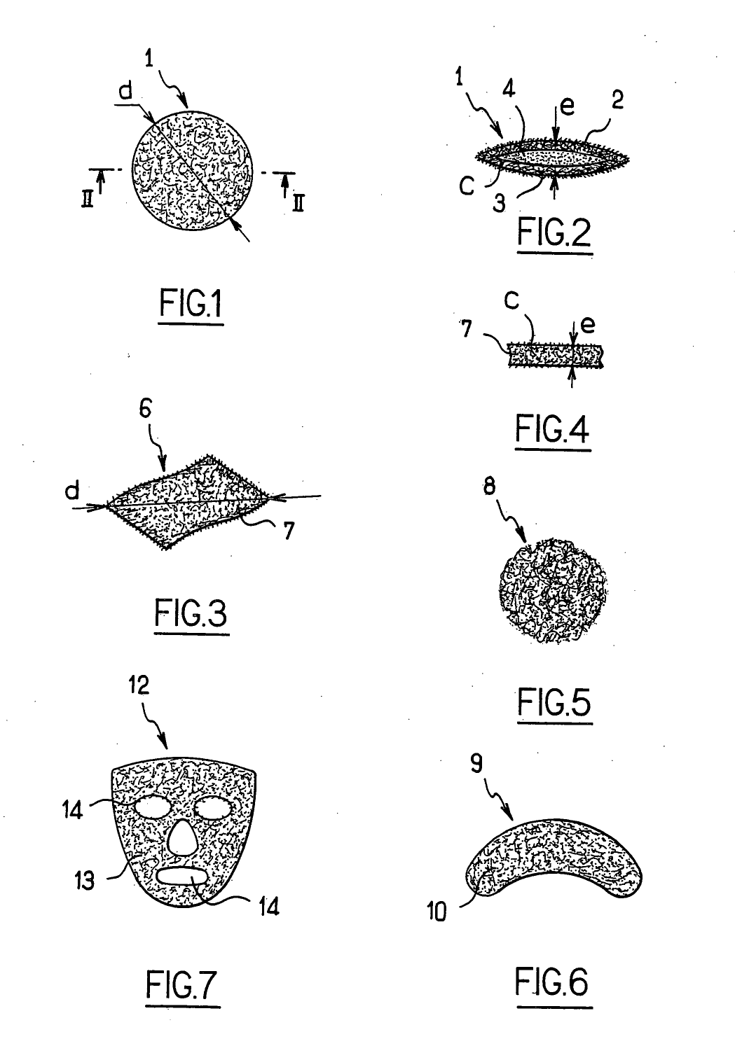

By way of example, the two layers are assembled together at their peripheries, as shown in FIGS. 1 and 2.

FIG. 1 is a plan view showing an article 1 comprising two sheets 2 and 3 of a non-woven fabric formed of water-soluble fibers, the two sheets being assembled together at their peripheries by heat-sealing, so as to constitute a cushion that is capable of retaining a cosmetic or dermatological composition C in an internal cavity 4, as can be seen in FIG. 2 (which is a cross section on II-II of FIG. 1).

The medium may also comprise only one layer of water-soluble fibers, and a cosmetic or dermatological composition may, for example, be dispersed within the layer, or may cover it on one of its surfaces only.

FIG. 3 is a diagrammatic and perspective view showing a cosmetic article 6 that comprises a single layer 7 formed of a non-woven fabric of soluble fibers, and in which the composition C is dispersed, said composition being in the form of a powder, for example, as shown in FIG. 4 (which is a fragmentary and diagrammatic cross-section of FIG. 3).

In FIG. 3, the article presents a rectangular shape, but the article could take some other shape, e.g. round or oval, having dimensions that enable it to be held between at least two fingers, for example. The article may form a wipe.

In the embodiments in FIGS. 1 and 3, the medium presents a generally flat shape, the maximum thickness e in FIGS. 2 and 4 being less than 10 mm, for example, for an area lying in the range 0.00005 square meters (m²) to 0.01 m², and preferably lying in the range 0.0001 m² to 0.001 m².

The medium may also present a shape that generally is not flat, presenting the appearance of a mass, as shown in FIG. 5, said figure being a plan view showing an article 8 formed of a globular mass of compacted water-soluble fibers incorporating the cosmetic or dermatological composition.

In an embodiment of the invention, the article formed by the cosmetic or dermatological composition and by the medium is intended to be put into contact with water before it is used. Thus, the medium is initially dissolved completely before the article is applied to the human body. Depending on the quantity of water that is added to the article in order to dissolve the medium, it is possible to adjust the apparent viscosity of the composition obtained.

In a variant embodiment of the invention, the article formed by the cosmetic or dermatological composition and by the medium is intended to be put into contact with water before it is used, but the medium is put into contact with a region of the human body, e.g. the skin, or hair, before it is completely dissolved, or even before it is wetted. Depending on the quantity of water that is added, this makes it possible, for example, to modify its properties as a function of the desired result. Water can be poured onto the article while said article is not in contact with the body. Alternatively, the body can be wet, or water can be sprayed or poured onto the medium while the article is in contact with the region to be treated.

In still another embodiment of the invention, the article is designed to be used without being impregnated with water. In this event, and if so desired by the user, the medium may be dissolved only after it has been used, by being disposed at the bottom of a washbowl, and by running water thereon, for example. By way of example, the article is pre-impregnated with the composition, or the user can deposit the composition thereon.

In particular, when the medium is not intended to be completely dissolved in water prior to application, the shape of the medium may depend on the region of the body to be treated.

FIG. 6 is thus a plan view showing an article 9 having a medium that is of kidney-shaped outline, and intended for applying makeup to the eyelids.

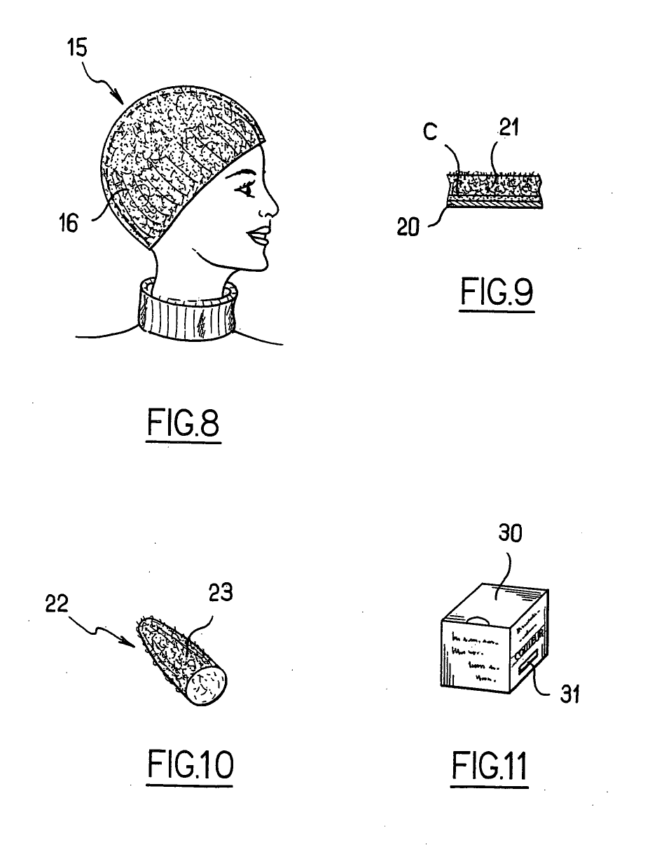

FIG. 7 is a front view showing an article 12 having a medium 13 that forms a mask, with openings or cutouts 14 for the eyes, the nose, and the mouth, and FIG. 8 is a diagram in perspective showing an article 15 having a medium 16 that forms a mobcap.

Cosmetic Articles Including a Medium that is not Completely Soluble in Water, in Particular in Water at Less than 20° C.

In another of its aspects, the invention also relates to a preferably flexible cosmetic or dermatological article comprising:
at least one medium presenting a multilayer structure with:
   at least one layer that is completely soluble in water, in particular at a temperature lower than 20° C.;
   at least one layer of a substrate that is insoluble in water, in particular at a temperature lower than 20° C.; and
at least one cosmetic composition carried by the medium.

By way of example, and as shown in FIG. 9 (which is a diagram in section showing an article according to this aspect of the invention), such a medium may comprise a layer 20 of a substrate that is insoluble in water, covered by a layer 21 constituted by fibers that are soluble in water, the cosmetic composition C being held, for example, between the layers 20 and 21, as shown. By way of example, the layers 20 and 21 are assembled together at their peripheries by heat-sealing or by some other means.

By way of example, the substrate 20 is a sheet of a non-woven synthetic fabric, e.g. a non-woven fabric of polyethylene, polypropylene, polyethylene terephthalate (PET), polylactic acid, polyamide, viscose, or cellulose fibers, or a mixture of these fibers . . . , or a film, that is optionally permeable. Non-woven fabrics are described in general manner in RIEDEL “Nonwoven Bonding Methods & Materials” Nonwoven World (1987), incorporated herein by reference.

By way of example, a multilayer structure with at least one layer formed by a substrate that is insoluble in water can be useful for making an article 22 including a medium 23 in the form of a finger of a glove, as shown in FIG. 10 (which is a diagram in perspective), such an article being for use in dental hygiene. The layer formed of water-soluble fibers is situated on the outside of the article since it is intended to dissolve while it is being used, after becoming wet or on coming into contact with a wet region of the body.

The layer 20 of insoluble substrate can also be in the form of a glove or of a mitten.

When the medium comprises a plurality of layers, the various layers may be assembled together in many ways, e.g. by heat-sealing, adhesive, or stitching, irrespective of whether or not said layers are all made out of water-soluble fibers, and where appropriate, the layers may constitute one or more cavities containing one or more cosmetic or dermatological compositions, or a plurality of components of a single cosmetic composition that are to be mixed together extemporaneously. During assembly by stitching, it is possible to use a thread that is itself water-soluble, where appropriate.

### Water-Soluble Fibers

As indicated above, the medium may comprise, or may even be constituted essentially by fibers that are soluble, preferably in water at less than 20° C.

By way of example, the medium may thus include more than 95% by weight, indeed more than 99% or even more, of water-soluble fibers.

The fibers are preferably made out of polyvinyl alcohol (PVA) by using a method that imparts the desired solubility thereto.

Fibers that are soluble in water at a temperature lower than 20° C. are sold by the Japanese company KURARAY under the trade name KURALON K-II WN2. The method of manufacturing such fibers includes the use of organic solvents. The fibers may be substantially circular in section.

European application EP 0 636 716, the content of which is incorporated herein by reference, describes PVA-based water-soluble fibers and their method of manufacture.

The invention is not limited to the use of PVA, and fibers made out of other water-soluble materials may be used, providing they dissolve in water at the desired temperature, e.g. polysaccharide fibers sold under the trade name LYSORB by LYSAC TECHNOLOGIES, INC, or starch hydrolysate polymer-based fibers such as glucomannan or starch.

Where appropriate, the medium may include a mixture of fibers that are soluble in water at various temperatures.

The fibers may be composite, and may, for example, comprise a core and a sheath that are not of the same type, e.g. that are formed of different grades of PVA.

The medium could have substantially no fibers that are insoluble in water.

In an aspect of the invention, the medium does not have any adhesive, in particular any pressure-sensitive adhesive.

### Cosmetic and Dermatological Compounds

Very diverse compounds may be used in an article made in accordance with the invention.

By way of example, they may be compounds used for makeup, for care, and/or for hygiene, and/or for cleaning the skin, the mouth, the lips, hair, or the nails.

The cosmetic or dermatological composition carried by the medium may represent between 10% and 1000% by weight relative to the weight of the medium, for example.

### Active Ingredients

The article may include at least one cosmetically or dermatologically active ingredient.

Examples of active ingredients that can be mentioned include antidandruff active ingredients that make it possible to clean excess sebum from the skin, and antimicrobial agents that eliminate micro-organisms that might be present on the skin, and mixtures thereof, this list not being limiting.

Examples of antidandruff active ingredients that can be mentioned include sulfur and sulfur derivatives, benzoyl peroxide, zinc derivatives such as zinc sulfate and zinc oxide, aluminum chloride, selenium disulfide, vitamins B and in particular panthenol (vitamin B5) and niacinamide (vitamin B6 or PP), and mixtures thereof.

Examples of antimicrobial agents that can be mentioned include: β-lactam derivatives, quinolone derivatives, ciprofloxacin, norfloxacin, tetracycline and salts thereof (hydrochloride), erythromycine and salts thereof (zinc, estolate, and stearate salts), amikacin and salts thereof (sulfate), 2,4,4′-trichloro-2′-hydroxy diphenyl ether (triclosan), 3,4,4′-trichlorobanilide (tricarban), phenoxyethanol, phenoxypropanol, phenoxyisopropanol, doxycycline and salts thereof (hydrochloride), capreomycin and salts thereof (sulfate), chlorhexidine and salts thereof (gluconate, hydrochloride), chlorotetracycline and salts thereof (hydrochloride), oxytetracycline and salts thereof (hydrochloride), clindamycin and salts thereof (hydrochloride), ethambutol and salts thereof (hydrochloride), hexamidine and salts thereof (isethionate), metronidazole and salts thereof (hydrochloride), pentamidine and salts thereof (hydrochloride), gentamicin and salts thereof (sulfate), kanamycin and salts thereof (sulfate), lineomycin and salts thereof (hydrochloride), methacycline and salts thereof (hydrochloride), methenamine and salts thereof (hippurate, mandelate), minocycline and salts thereof (hydrochloride), neomycin and salts thereof (sulfate), netilmicin and salts thereof (sulfate), paromomycine and salts thereof (sulfate), streptomycin and salts thereof (sulfate), tobramycin and salts thereof (sulfate), miconazole and salts thereof (hydrochloride), amanfadine and salts thereof (sulfate, hydrochloride), octopirox, parachlorometaxylenol, nystatin, tolnaftate, zinc pyrithione, clotrimazole, salicylic acid, n-octanoyl-5 salicylic acid (or capryloylsalicylic acid), benzoyl peroxide, 3-hydroxybenzoic acid, glycolic acid, lactic acid, 4-hydroxybenzoic acid, acetylsalicylic acid, 2-hydroxybutanoic acid, 2-hydroxypentanoic acid, 2-hydroxyhexanoic acid, phytoic acid, N-acetyl-L-cysteine acid, lipoic acid, azelaic acid, arachidonic acid, ibuprofen, naproxen, hydrocortisone, acetominophen, resorcinol, lidocaine hydrochloride, neocycin sulfate, octoxyglycerin, octanoylglycine (or capryloyl glycine), caprylyl glycol (1,2-octanediol), 10-hydroxy-2-decanoic acid, and mixtures thereof. The preferred antimicrobial agents are 2,4,4′-trichloro-2′-hydroxy diphenyl ether, 3,4,4′-trichlorobanilide, phenoxyethanol, phenoxypropanol, phenoxyisopropanol, chlorhexidine and salts thereof, octopirox, zinc pyrithione, salicylic acid, n-octanoyl-5 salicylic acid, benzoyl peroxide, 3-hydroxybenzoic acid, glycolic acid, lactic acid, 4-hydroxybenzoic acid, acetylsalicylic acid, 2-hydroxybutanoic acid, 2-hydroxypentanoic acid, 2-hydroxyhexanoic acid, phytic acid, N-acetyl-L-cysteine acid, lipoic acid, azelaic acid, arachidonic acid, octoxyglycerin, octanoyl glycine, caprylyl glycol, 10-hydroxy-2-decanoic acid, and mixtures thereof.

The composition may comprise active ingredients acting on the barrier function of the skin, active ingredients favoring the moisturizing of the skin, and peeling agents.

The term “peeling agent” means any compound that is capable of acting:
either directly on peeling, favoring exfoliation, such as β-hydroxy acids, in particular salicylic acid and derivatives thereof (including n-octanoyl 5-salicylic acid); α-hydroxy acids, such as glycolic, citric, lactic, tartric, malic, or mandelic acids; urea; gentisic acid; oligofucoses; cinnamic acid; Saphora japonica extract; resveratrol;
on the enzymes implicated in flaking or in degrading the corneodesmosomes, such as glycosidases, stratum corneum chymotryptic enzyme (SCCE), or other proteases (trypsin, chymotrypsin-like). Mention can be made of inorganic salt chelating agents: EDTA; N-acyl-N,N′,N′ ethylenediamine-triacetic acid; aminosulfonic compounds, and in particular N-(2-hydroxyethyl)piperazine-N′-2-ethane sulfonic acid (HEPES); derivatives of 2-oxothiazolidine-4-carboxylic acid (procysteine); derivatives of glycine-type alpha amino acids (as described in EP-0 852 949, together with methylglycine sodium diacetate sold by BASF under the trade name “TRILON M”); honey; sugar derivatives such as O-octanoyl-6-D-maltose and N-acetyl glucosamine.

Amongst the active ingredients acting on the barrier function of the skin or favoring the moisturizing of the skin, mention can be made of:
compounds acting on the barrier function with a view to keeping the stratum corneum moisturized, or occlusive compounds, in particular ceramids, sphingoid-based compounds, lecithins, glycosphingolipids, phospholipids, cholesterol and derivatives thereof, phytosterols (stigmasterol, β-sitosterol, campesterol), essential fatty acids, 1-2 diacylglycerol, 4-chromanone, pentacyclic triterpenes such as ursolic acid, vaseline and lanolin;
compounds directly increasing the water content of the stratum corneum, such as threalose and derivatives thereof, hyaluronic acid and derivatives thereof, glycerol, pentanediol, sodium pidolate, serine, xylitol, sodium lactate, glycerol polyacrylate, ectoine and derivatives thereof, chitosan, oligo- and poly-saccharides, cyclic carbonates, N-lauroyl pyrrolidone carboxylic acid, and N-α-benzoyl-L-arginine.

Suitable cosmetically, dermatologically, or hygienically active ingredients that can also be mentioned are vitamins (C, A, E, F, B, or PP), essential fatty acids, essential oils, sun screens, in particular liposoluble or nanoparticle sun screens, specific skin treatment active ingredients (anti-odor agents, anti-wrinkle agents, . . . ), self-tanning agents, any plant, mineral, or marine extracts, antiradical or soothing substances, and slimming substances (caffeine, . . . ). All of said active ingredients may be used in concentrations in the range 0 to 20%, for example, and in particular in the range 0.001% to 15% relative to the total weight of the composition.

All or part of the active ingredients may be encapsulated, where appropriate.

### Coloring Agents

The article may include at least one coloring agent in a quantity lying in the range 0 to 100%, e.g. in the range 0 to 90%, preferably in the range 0.1% to 50%, and better in the range 1% to 20% of the total weight of the composition, and in particular at least one pigment or colorant, e.g. a coloring agent selected from inorganic pigments, organic lakes or pigments, nacre pigments, composite pigments, and liposoluble and water-soluble colorants.

The inorganic pigments may optionally be coated. Mention can be made of titanium dioxide that is optionally surface treated, zirconium or cerium oxides, together with iron or chromium oxides, manganese violet, ultramarine, chromium hydrate, and iron blue.

Nacre pigments may be selected from white nacre pigments such as mica coated in titanium or bismuth oxychloride; colored nacre pigments such as mica titanium with iron oxides, mica titanium with iron blue or chromium oxide in particular; gold color nacres such as those sold by ENGELHARD under the trade names Brillant gold 212G (Timica), Gold 222C (Cloisonne), Sparkle gold (Timica), Gold 4504 (Chromalite), and Monarch gold 233X (Cloisonne); bronze nacres, in particular those sold by MERCK under the trade names Bronze fine (17384) (Colorona) and Bronze (17353) (Colorona), and by ENGELHARD under the trade name Super bronze (Cloisonne); orange nacres especially those sold by ENGELHARD under the trade names Orange 363C (Cloisonne) and Orange MCR 101 (Cosmica), and by MERCK under the trade names Passion orange (Colorona) and Matte orange (17449) (Microna); brown-tinted nacres sold by ENGELHARD under the trade names Nu-antique copper 340XB (Cloisonne) and Brown CL4509 (Chromalite); nacres with a copper glint sold by ENGELHARD under the trade name Copper 340A (Timica); nacres with a red glint, especially those sold by MERCK under the trade name Sienna fine (17386) (Colorona); nacres with a yellow glint, especially those sold by ENGELHARD under the trade name Yellow (4502) (Chromalite); red-tinted nacres with gold glints, especially those sold by ENGELHARD under the trade name Sunstone G012 (Gemtone); pink nacres, especially those sold by ENGELHARD under the trade name Tan opale G005 (Gemtone); black nacres with a glint, especially those sold by ENGELHARD under the trade name Nu antique bronze 240 AB (Timica); blue nacres, especially those sold by MERCK under the trade name Matte blue (17433) (Microna); white nacres with silvery glints, especially those sold by MERCK under the trade name Xirona Silver; and orange-pink green-gold highlight nacres sold by MERCK under the trade names Indian summer (Xirona) and mixtures thereof. Examples of liposoluble colorants are plant extracts, Sudan red, D&C Red No. 17, D&C Green No. 6, β-carotene, soybean oil, Sudan brown, D&C Yellow No. 11, D&C Violet No. 2, D&C orange No. 5, and quinoline yellow.

The water-soluble colorants are, for example, selected from plant extracts, in particular beet juice and methylene blue.

The coloring agent may comprise at least one organic coloring substance, e.g. at least one organic pigment and/or at least one organic lake.

By way of example, the organic coloring substance may comprise organic lakes or pigments that may be selected from the following compounds and mixtures thereof:
cochineal carmine;
the organic pigments of azo, anthraquinone, indigo, xanthene, pyrene, quinoline, triphenylmethane, or fluorane dyes;
organic lakes or insoluble salts of sodium, potassium, calcium, barium, aluminum, zirconium, strontium, titanium, or of acid dyes such as azo, anthraquinone, indigo, xanthene, pyrene, quinoline, triphenylmethane, or fluorine dyes, which dyes may comprise at least one carboxylic or sulfonic acid group.

Organic pigments that can be mentioned include those with the following denominations: D&C Blue No. 4, D&C Brown No. 1, D&C Green No. 5, D&C Green No. 6, D&C Orange No. 4, D&C Orange No. 5, D&C Orange No. 10, D&C Orange No. 11, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Violet No. 2, D&C Yellow No. 7, D&C Yellow No. 8, D&C Yellow No. 10, D&C Yellow No. 11, FD&C Blue No. 1, FD&C Green No. 3, FD&C Red No. 40, FD&C Yellow No. 5, FD&C Yellow No. 6.

The organic coloring substance may comprise an organic lake supported by an organic medium such as colophane or aluminum benzoate, for example.

Particular organic lakes that can be mentioned include those with the following denominations: D&C Red No. 2 Aluminum lake, D&C Red No. 3 Aluminum lake, D&C Red No. 4 Aluminum lake, D&C Red No. 6 Aluminum lake, D&C Red No. 6 Barium lake, D&C Red No. 6 Barium/Strontium lake, D&C Red No. 6 Strontium lake, D&C Red No. 6 Potassium lake, D&C Red No. 7 Aluminum lake, D&C Red No. 7 Barium lake, D&C Red No. 7 Calcium lake, D&C Red No. 7 Calcium/Strontium lake, D&C Red No. 7 Zirconium lake, D&C Red No. 8 Sodium lake, D&C Red No. 9 Aluminum lake, D&C Red No. 9 Barium lake, D&C Red No. 9 Barium/Strontium lake, D&C Red No. 9 Zirconium lake, D&C Red No. 10 Sodium lake, D&C Red No. 19 Aluminum lake, D&C Red No. 19 Barium lake, D&C Red No. 19 Zirconium lake, D&C Red No. 21 Aluminum lake, D&C Red No. 21 Zirconium lake, D&C Red No. 22 Aluminum lake, D&C Red No. 27 Aluminum lake, D&C Red No. 27 Aluminum/Titanium/Zirconium lake, D&C Red No. 27 Barium lake, D&C Red No. 27 Calcium lake, D&C Red No. 27 Zirconium lake, D&C Red No. 28 Aluminum lake, D&C Red No. 30 lake, D&C Red No. 31 Calcium lake, D&C Red No. 33 Aluminum lake, D&C Red No. 34 Calcium lake, D&C Red No. 36 lake, D&C Red No. 40 Aluminum lake, D&C Blue No. 1 Aluminum lake, D&C Green No. 3 Aluminum lake, D&C Orange No. 4 Aluminum lake, D&C Orange No. 5 Aluminum lake, D&C Orange No. 5 Zirconium lake, D&C Orange No. 10 Aluminum lake, D&C Orange No. 17 Barium lake, D&C Yellow No. 5 Aluminum lake, D&C Yellow No. 5 Zirconium lake, D&C Yellow No. 6 Aluminum lake, D&C Yellow No. 7 Zirconium lake, D&C Yellow No. 10 Aluminum lake, FD&C Blue No. 1 Aluminum lake, FD&C Red No. 4 Aluminum lake, FD&C Red No. 40 Aluminum lake, FD&C Yellow No. 5 Aluminum lake, FD&C Yellow No. 6 Aluminum lake.

The compounds corresponding to each of the organic coloring substances listed above are mentioned in the work entitled “International Cosmetic Ingredient Dictionary and Handbook”, 1997 edition, pages 371 to 386 and 524 to 528, published by “The Cosmetic, Toiletry, and Fragrance Association”, the contents of which are incorporated herein by reference.

The article may include at least one goniochromatic coloring agent.

The goniochromatic coloring agent may, for example, be selected from the multilayer interference structures and the liquid crystal coloring agents, e.g. Fe₂O₃/SiO₂/Fe₂O₃/SiO₂/Fe₂O₃, a pigment having this structure being sold under the tradename SICOPEARL by BASF; MoS₂/SiO₂/mica-oxide/SiO₂/MoS₂; Fe₂O₃/SiO₂/mica-oxide/SiO₂/Fe₂O3; TiO₂/SiO₂/TiO₂ or TiO₂/Al₂O3/TiO₂, pigments with these structures being sold under the tradename XIRONA by MERCK (Darmstadt).

The coloring agent may also comprise a diffractive pigment presenting the structure MgF₂/Al/MgF₂, for example, as sold under the trade names SPECTRAFLAIR 1400 Pigment Silver by FLEX PRODUCTS, or SPECTRAFLAIR 1400 Pigment Silver FG.

Regardless of the nature of the pigments used, the article may comprise not only the particulate phase comprising the pigments, but also at least one binder comprising at least one hydrophilic solid elastomeric organopolysiloxane. Examples of such polymers are given in US application No. 2004/0071648, the contents of which are incorporated herein by reference.

### Film-Forming Agents

The article may include one or more film-forming agents, e.g. when it is intended for applying makeup or for forming a peel-off film or a skin-care mask.

The medium itself may serve as a film-forming agent once it has dissolved, in particular when the medium is made out of PVA.

The medium may thus be provided in a quantity that is sufficient to enable a continuous film to be formed on keratinous materials.

The article may include other film-forming agents, which should be selected as a function of the nature of the medium, for example.

The other film-forming agents may be selected from synthetic polymers of the radical or polycondensate type, polymers of natural origin, and mixtures thereof.

The radical type film-forming polymers may, for example, be vinyl polymers or copolymers, in particular acrylic polymers.

The vinyl film-forming polymers may be obtained by polymerizing monomers with an ethylenically unsaturated bond containing at least one acid group and/or esters of said acid monomers and/or amides of said acid monomers, such as α,β-ethylenically unsaturated carboxylic acids.

Examples of film-forming polycondensates that can be mentioned include polyurethanes, polyesters, polyester amides, polyamides, and polyureas, this list not being limiting.

Polymers of natural origin, which may optionally be modified, may be selected from shellac resin, gum sandarac, dammar resin, gum elemi, copal resins, cellulose polymers.

### Other Ingredients

The article may also contain any ingredients that are routinely used in cosmetics or dermatology, and that are physiologically acceptable, such as fillers, thickeners, surfactants, oligo-elements, sequestrating agents, fragrances, alkalinizing or acidifying agents, preservatives, and antioxidants, for example.

By way of example, the article may include at least one compound for enabling lather to be formed when the article is put into contact with water and stirred, e.g. at least one anionic, cationic, amphoteric, or non-ionic surfactant. Examples of this type of surfactants of that can be mentioned include:

(1) amongst non-ionic surfactants, oxypropylene/oxyethylene block polymers such as Poloxamer 184 (Cosmetic, Toiletry, and Fragrance Association (CTFA) name); alkyl polyglycosides, and in particular alkyl polyglucosides (APG) having an alkyl group of 6 to 30 carbon atoms (C₆-C₃₀-alkyl polyglucosides), and preferably 8 to 16 carbon atoms, such as decyl glucoside (C9/C11-alkyl polyglucoside (1.4), for example, such as the product sold under the trade name MYDOL 10 by Kao Chemicals, the product sold under the trade name PLANTAREN 2000 UP or PLANTACARE 2000 UP by Henkel, and the product sold under the trade name ORAMIX NS 10 by Seppic; caprylyl/capryl glucoside such as the product sold under the trade name ORAMIX CG 110 by Seppic; lauryl glucoside such as the products sold under the trade names PLANTAREN 1200 N and PLANTACARE 1200 by Henkel; and coco-glucoside such as the product sold under the trade name PLANTACARE 818/UP by Henkel;

(2) amongst anionic surfactants, alkyl sulfates, alkyl ether sulfates and their salts, in particular their sodium salts, such as the mixture of Sodium Laureth Sulfate/Magnesium Laureth Sulfate/Sodium Laureth-8 Sulfate/Magnesium Laureth-8 Sulfate, sold under the trade name Texapon ASV by Henkel; sodium lauryl ether sulfate (C12-14 70/30) (2,2 OE) sold under the trade names SIPON AOS 225 or TEXAPON N702 PATE by Henkel, ammonium lauryl ether sulfate (C12-14 70/30) (3 OE) sold under the tradename SIPON LEA 370 by Henkel; ammonium alkyl ether sulfate (C12-C14) (9 OE) sold under the trade name RHODAPEX AB/20 by Rhodia Chimie;

(3) amongst amphoteric or zwitterionic surfactants, alkylamido alkylamine derivatives such as N-cocoyl-N-carboxymethoxyethyl-N-carboxymethyl-ethylenediamine N-disodic (CTFA name: disodium cocoampho-diacetate) sold in aqueous saline solution under the trade name MIRANOL C2M CONC NP by Rhodia Chimie; N-cocoyl-N-hydroxyethyl-N-carboxymethyl-ethylenediamine N-sodic (CTFA name: sodium cocampho-acetate); and the mixture of coco acid ethanolamide (CTFA name: Cocamide DEA).

The article may also include a mixture of said surfactants.

The article may include compounds that are intended to react together in the presence of water, e.g. to cause effervescence, e.g. at least one organic acid, in particular citric acid, and at least one alkaline agent, e.g. bicarbonate of soda.

### Ways of Incorporating the Cosmetic or Dermatological Compound(s) in the Article

At least one cosmetic or dermatological compound, and more generally the cosmetic or dermatological composition, may optionally be present on the outside of the article.

When the composition is present on the outside of the article, said article can be used by being brought into contact with the region to be treated, for example, so as to enable the composition to be transferred. Where appropriate, said transfer can be performed by rubbing the article on the region to be treated.

When the composition is present on the outside of the article, said article need not be wet while it is being used, and the medium is dissolved only after it has been used.

When the composition is present on the outside of the medium, the article may, in some cases, nevertheless be wet while it is being used, e.g. so as to modify the properties of the composition, improve the transfer of the composition, or form a film-forming substance.

When the composition is not present on the outside of the article, it may be necessary to dissolve the medium, at least in part, or even completely, before use.

At least part and possibly even all of the composition may be present inside the article in powder form, in particular in the form of a substantially anhydrous powder. The article may be dry to the touch, for example. The grain size of the composition should be selected as a function of the porosity of the medium so as to reduce losses when shaken.

When the composition is a powder, it may be contained in a cavity in the medium, e.g. formed between two layers thereof, as in the embodiment in FIG. 2, or it may be dispersed, e.g. in uniform manner within the medium, in particular within a single fibrous layer of the medium, as in the embodiment in FIG. 4.

The composition need not be a powder but could be a creamy or pasty liquid forming a paste, for example. In this event, the composition may include one or more solvents, for example, that are compatible with the nature of the medium, in particular non-aqueous solvents, so as to prevent it from dissolving prematurely, such as inferior alcohols including 1 to 6 carbon atoms, such as ethanol; polyols such as glycerine; glycols such as butylene glycol, isoprene glycol, hexylene glycol, propylene glycol; polyethylene glycols, such as PEG-8, sorbitol; animal hydrocarbon oils, such as perhydrosqualene; vegetable hydrocarbon oils, such as glycerol or fatty acid mono-, di-, or tri-ester glycerides, e.g. sweet almond, sunflower, corn, soybean, nut, or apricot kernel oils; synthetic triglycerides, such as those sold by Stearineries Dubois, or those sold under the trade name Miglyol 810, 812, and 818 by Dynamit Nobel; synthesized esters and ethers, in particular fatty acids, such as oils of formulae R¹COOR² et R¹OR² in which R¹ represents the fatty acid residue including 8 to 29 carbon atoms, and R² represents an optionally-branched hydrocarbon chain containing 3 to 30 carbon atoms, such as Purcellin oil, isononyl isononanoate, 2-octyldodecyl stearate, isostearyl lactate, octylhydroxystearate, or tri-isocetyl citrate, for example; polyol esters, such as propylene glycol dioctanoate, diethyleneglycol di-isononanoate; and pentaerythritol esters; linear or branched hydrocarbons of mineral or synthetic origin, such as volatile or non-volatile paraffin oils; fatty alcohols having 8 to 26 carbon atoms, such as cetylic alcohol, stearyl alcohol, and mixtures thereof (cetearyl alcohol), octyldodecanol, 2-hexyldecanol; alkoxylated fatty alcohol, and in particular ethoxylated fatty alcohol, such as oleth-12 or ceteareth-20; partially fluorinated hycrocarbon and/or silicone oils such as those described in document JP-A-2-295912. Suitable fluorinated oils that can also be mentioned are perfluoromethylcyclopentane and 1,3-perfluorodimethylcyclohexane, sold under the trade names “FLUTEC PC1®” and “FLUTEC PC3®” by BNFL Fluorochemicals; perfluoroalkanes such as dodecafluoropentane and tetradecafluorohexane, sold under the trade names “PF 5050®” and “PF 5060®” by 3M; silicone oils such as volatile or non-volatile polymethylsiloxanes (PDMS) having a linear or cyclic silicone chain, which may be liquid or pasty at ambient temperature, in particular cyclopolydimethylsiloxanes (cyclomethicones) such as cyclohexasiloxane; polydimethylsiloxanes comprising alkyl, alcoxy, or phenyl groups, pendant or at the end of a silocone chain, groups having 2 to 24 carbon atoms; phenyl silicones such as phenyltrimethicones, les phenyldimethicones, phenyltrimethylsiloxydiphenyl-siloxanes, diphenyl-dimethicones, diphenylmethyldiphenyl trisiloxanes, 2-phenylethyltrimethyl-siloxysilicates, and polymethylphenylsiloxanes.

The term “hydrocarbon oil” in the list of oils mentioned above means any oil containing mainly hydrogen and carbon atoms, and optionally the following groups: ester, ether, fluorine, carboxylic acid, and/or alcohol.

The composition could contain a certain quantity of water when it is impregnated in the medium. In order to prevent it from dissolving prematurely, the water that is introduced into the medium during its impregnation is eliminated by conventional means used to dehydrate compositions containing water, e.g. by heating.

The composition may be deposited on only one surface of the medium, the other surface of the medium being used for holding the article, for example.

For a colored composition, the article is packaged in a packaging such as a box 30, for example, as shown in FIG. 11. Where appropriate, an indicator 31 indicating the color of the substance that is obtained after the medium has dissolved may be displayed on the packaging, so as to inform the consumer before purchase.

When the composition is incorporated in the article during its manufacture, the article is packaged loosely in a box or in individual packaging. Where appropriate, the articles are packaged in a string. The articles may also be folded in half and interfitted, so that removing one article brings the next into a configuration making it easy to grasp.

When the composition must be deposited on the medium by the user, the composition and the medium may be proposed in the form of a kit, for example. By way of example, the composition is supplied in a quantity that is sufficient to enable a plurality of doses to be dispensed on a set of mediums that are intended to be used successively.

### EXAMPLES

### Applications in Applying Makeup/Compositions that are Transferable Merely by Making Contact with or Rubbing Against the Skin, the Lips, or Hair

### Example A

### Applying Makeup to the Skin, in Particular to the Eyelids

A medium is made by cutting out an egg shape, that is about 5 cm long and 1.5 cm wide, from a sheet of KURALON K-II WN2 that is about 0.5 mm thick and that weighs 60 grams per square meter (g/m²).

Depending on the desired intensity of the makeup, the medium is impregnated with a quantity, lying in the range 0.02 g to 0.1 g, of the following colored powder composition, deposited on a surface thereof.

| | |
|---|---|
| Mica | 14% |
| Ferric ferrocyanide | 6% |
| Talc | 8% |
| Calcium sodium borosilicate | 60% |
| Titanium dioxide | 10% |
| Ethylhexyl palmitate | 1.5% |
| Preservatives | 0.5% |

The medium impregnated in this way is soft to the touch and easy to hold, in particular by its surface that is opposite from the surface carrying the composition, and it can be displaced on the skin so as to enable the composition to be transferred thereto.

After use, the medium is easily eliminated under running water.

The medium can be impregnated while it is being used, or the medium can be proposed to the user pre-impregnated, in particular in an individual packaging.

### Example B

### Applying Makeup to the Skin, in Particular to the Cheeks

A medium is made by cutting out a round shape, that is about 5 cm in diameter, from a sheet of KURALON K-II WN2 that is about 2 mm thick and that weighs 80 g/m².

The medium is impregnated with 0.1 g of the following powder composition, deposited on a surface thereof.

| | |
|---|---|
| Mica | 30% |
| Pigments of iron oxides and of zinc | 3.5% |
| oxides | |
| Talc | 35% |
| Titanium dioxide | 5% |
| Nylon 12 | 20% |
| Ethylhexyl palmitate | 2% |
| Mineral oil | 4% |
| Preservatives | 0.5% |

After use, the medium is easily eliminated under running water.

### Example C

### Applying Makeup to the Skin, in Particular to the Face

The same medium as in Example A is coated in 0.25 g of the following anhydrous cream-blusher composition:

| | |
|---|---|
| Mica | 2% |
| Pigments based on iron oxides | 3% |
| Talc | 4% |
| Nylon 12 | 2% |
| Hydrogenated styrene/isoprene copolymer | 10% |
| Isohexadedane | 25% |
| Disteardimonium hectorite | 2% |
| Silica | 5% |
| Isononyl isononanoate | 15% |
| Polymethyl methacrylate | 15% |
| Petroleum distillates | 17% |

After use, the medium is easily eliminated by dissolving in cold water.

### Applications in Applying Makeup/Articles Having a Medium that is Dissolved Before being Applied

### Example D

### Applying Makeup to the Skin

An article such as the article shown in FIGS. 1 and 2 is made out of KURALON K-II WN2 fibers by heat-sealing the layers 2 and 3 at their peripheries, after previously inserting the cosmetic composition into the cavity 4. The layers 2 and 3 are of weight lying in the range 70 g/m² to 80 g/m², and of thickness lying in the range 3 mm to 4 mm. The article is in the form of a disk that is 3 cm in diameter, and containing about 0.3 g of a colored powder composition having the following formulation:

| | |
|---|---|
| Titanium oxide (untreated anatase) | 20% |
| Yellow iron oxide | 4.5% |
| Yellow brown iron oxide | 4% |
| Black iron oxide | 1% |
| Nylon powder (Orgasol from Atochem) | 30% |
| Dimethicone/Vinyl dimethicone | 35.5% |
| crosspolymer | |
| Glycerol | 4.5% |
| Preservatives | 0.5% |

In order to use the article, said article is disposed in the hollow of the hand and water is poured onto the medium.

The PVA fibers contribute to texture the composition and/or impart film-forming properties thereto. The user can influence the texture of the substance by adding more or less water.

The substance formed in this way can be spread directly onto the body or the face by means of the fingers or by using an applicator, e.g. a flocked endpiece, such as a foam, a felt, or cotton wool, or a brush.

### Applications for Hygiene and/or Care Purposes

### Example E

### Under-Arm Hygiene

An article is made comprising a medium constituted by a disk, that is 7 cm in diameter, of a 2 mm thick sheet of KURALON K-II WN2 fibers weighing 80 g/m², and that is impregnated with 0.2 g of a powder deodorant composition having the following formulation:

| | |
|---|---|
| Cetyl stearyl alcohol | 2% |
| Fragrance | 0.5% |
| Preservatives | 0.5% |
| Polydimethylsiloxane | 0.5% |
| Aluminum hydroxychloride | 96.5% |

The article is used dry by wiping over the armpits.

The affinity of the medium for water makes it possible to wipe away any possible residues of transpiration.

### Example F

### Cleaning the Skin

An article such as the article shown in FIGS. 1 and 2 is made out of KURALON K-II WN2 fibers by heat-sealing the layers 2 and 3 at their peripheries, after previously inserting the cosmetic composition into the cavity 4. The article is in the form of a disk that is 3 cm in diameter, and the article contains about 0.3 g of a powder cleaning composition having the following formulation:

| | |
|---|---|
| Starch (octenyl succinyl starch) | 40% |
| Sodium cocoyl isethionate | 34.9% |
| Potassium laurate | 10% |
| Potassium myristate | 10% |
| Fragrance | 3% |
| Salicyclic acid | 2% |
| Preservatives | 0.1% |

In order to use the article, said article is disposed in the hollow of the hand and about 3 cubic centimeters (cm³) of water is poured thereon.

Said article is stirred by means of the index finger of the other hand, adding up to 3 cm³ to 6 cm³ of water if necessary, so as to obtain a uniform lather after the medium has completely dissolved.

The lather can be used to wash the hands or the face, for example.

### Example G

### Cleaning the Skin

A wipe is made as follows:

A square sheet having sides of 10 cm, a weight of 60 g/m², and a thickness of 1 mm, is cut out of KURALON K-II WN2, and is impregnated with 0.9 g of the following concentrated latherable composition:

| | |
|---|---|
| 70% sodium laureth sulfate in water | 50% |
| (Texapon N702 from Cognis) | |
| Disodium cocoamphodiacetate at 39% in | 24.9% |
| briny water (11% sodium chloride) | |
| Miranol C2M from Rhodia | |
| Sodium lauroyl sarcosinate at 90% in | 16.9% |
| water (Sarkosyl NL97 from Ciba Geigy) | |
| Fragrance | 3% |
| Glycerol | 5% |
| Preservatives | 0.3% |

This composition contains at least 15% water. It is in the form of a paste that is deposited on the medium using conventional means, such as rolling, for example.

The composition is dried, and once dry said composition adheres to the fibers.

Once dry, the medium is calendered between rollers so as to densify it, stiffen it, and reduce its surface porosity.

In order to use the article, the wipe is passed under water, then rubbed so as to create the lather and dissolve the medium.

### Example H

### Shaving the Skin

An article for use in shaving is made with the same medium as that in Example E, the composition of Example E being replaced as follows:

| | |
|---|---|
| Stearic acid | 10% |
| Palmitic acid | 10% |
| Myristic acid | 10% |
| Dimethiconol stearate | 0.5% |
| Disodium cocoamphodiacetate | 5% |
| Potassium hydroxide | 8% |
| Water | 51.2% |
| Glycerol | 5% |
| Preservatives | 0.3% |

This composition contains at least 15% water. It is in the form of a paste that is deposited on the medium using conventional means, such as rolling, for example.

The composition is dried, and once dry said composition adheres to the fibers.

Once dry, the medium is calendered between rollers so as to densify it, stiffen it, and reduce its surface porosity.

In order to use the article, said article is applied to the already moistened cheeks, then it is rubbed while gradually adding water.

### Example I

### Removing Makeup

A makeup-remover compress for removing makeup from the face is made with the same medium as that in Example E, the composition of Example E being replaced as follows:

| | |
|---|---|
| Parleam oil | 40% |
| Isohexadecane | 28% |
| Dextrine palmitate (Rheopearl TL) | 2% |
| PEG-20 glyceryl tri-isostearate | 25% |
| Simulgel 600 (CTFA name: Acrylamide/ | 5% |
| Sodium acryloyldimethyltaurate | |
| copolymer/Isohexadedane/Polysorbate 80) | |

The composition is anhydrous, it is impregnated into the medium using conventional means, such as applicator rollers or impregnation strips, for example.

A makeup remover emulsion is generated by passing the sheet over the wet face, or better by rubbing it in the hollow of the hand with a small amount of water.

Naturally, the invention is not limited to the examples given above.

In particular, active ingredients other than those mentioned above can be used.

Throughout the description, including in the claims, the proportions are proportion by weight, unless specified to the contrary.

The expression “comprising a” should be understood as being synonymous with “comprising at least one” unless specified to the contrary.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

The expression “in the range” should be understood as including the limits of the range.

What is claimed is:

1. A method for cosmetic treatment of keratinous material comprising:
providing a water soluble substrate having a density not greater than 0.1 g/cm³ and comprising:
a material that is soluble in water at a temperature lower than 20° C.; and
at least one foaming cosmetic compound;
forming a cosmetic composition by contacting said substrate with a sufficient quantity of water in order to substantially dissolve the substrate and causing the foaming cosmetic compound to foam;
applying the thus formed composition formed to the keratinous material.

2. A method according to claim 1, wherein the article is flexible.

3. A method according to claim 1, wherein the article is not adhesive.

4. A method according to claim 1, wherein the substrate is completely soluble in water.

5. A method according to claim 1, wherein the substrate includes fibers that are soluble in water.

6. A method according to claim 5, wherein the fibers are soluble at a temperature lower than 20° C.

7. A method according to claim 5, wherein the substrate is constituted essentially by said soluble fibers.

8. A method according to claim 1, wherein the substrate comprises at least one layer of a non-woven fabric that is constituted essentially by fibers that are soluble in water.

9. A method according to claim 8, wherein the fibers are soluble at a temperature lower than 20° C.

10. A method according to claim 1, wherein the substrate comprises at least two layers of non-woven fabric that are each constituted essentially by fibers that are soluble in water.

11. A method according to claim 10, wherein the fibers are soluble at a temperature lower than 20° C.

12. A method according to claim 10, wherein the two layers comprise two sheets that are assembled together at their peripheries.

13. A method according to claim 12, wherein the sheets are heat-sealed together.

14. A method according to claim 2, wherein the compound is dispersed, at least in part, within the substrate.

15. A method according to claim 10, wherein the compound is disposed at least in part between the two layers.

16. A method according to claim 1, wherein the article comprises a generally flat shape.

17. A method according to claim 1, wherein the article comprises a generally globular shape.

18. A method according to claim 1, further comprising at least one layer that is constituted essentially by fibers that are soluble in water and at least one layer of a substrate that is insoluble in water.

19. A method according to claim 18, wherein the fibers are soluble at a temperature lower than 20° C.

20. A method according to claim 5, wherein the fibers comprise polyvinyl alcohol.

21. A method according to claim 20, in which the fibers include a solid cross-section that is substantially circular.

22. A method according to claim 1, wherein the cosmetic compound comprises at least one active ingredient for at least one of a care purpose and a hygiene purpose.

23. A method according to claim 1, wherein the cosmetic compound comprises a makeup composition, for making up at least one of skin, lips, hair, and nails.

24. A method according to claim 1, wherein the cosmetic compound comprises a composition for hair.

25. A method according to claim 1, wherein the article is configured to be capable of forming a continuous film that is configured to be peeled off once the substrate has dissolved.

26. A method according to claim 1, wherein the compound comprises at least one hydrophilic solid elastomeric organopolysiloxane.

27. A method according to claim 1, wherein the substrate comprises at least one of a cushion, a mask, a patch, a mobcap, a glove, a finger of a glove, a cut-out sheet, a wipe, a disk, an oval, or a rectangle.

28. A method according to claim 1, wherein the article is dry to touch before use.

29. A method according to claim 1, wherein the compound comprises a composition that represents between 10% and 1000% by weight relative to the weight of the substrate.

30. A method according to claim 1, wherein the water soluble substrate is placed in a hollow of a hand when it is contacted with water.
